(19) United States
(12) Patent Application Publication
(10) Pub. No.: US 2022/0340975 A1
(43) Pub. Date: Oct. 27, 2022

(54) METHOD OF TREATMENT AND PRONOSTIC OF ACUTE MYELOID LEUKEMIA

(71) Applicants: INSERM (Institute National de la Santé et de la Recherche Médicale), Paris (FR); Centre National de la Recherche Scientifique (CNRS), Paris (FR); Université d'Aix Marseille, Marseille Cedex 07 (FR); Institut Jean Paoli & Irene Calmettes, Marseille (FR)

(72) Inventors: Norbert VEY, Marseille Cedex 09 (FR); Sylvain GARCIAZ, Marseille (FR); Estelle DUPREZ, Marseille (FR); Dasi-Lia N'GUYEN, Marseille (FR)

(21) Appl. No.: 17/637,235

(22) PCT Filed: Sep. 4, 2020
(86) PCT No.: PCT/EP2020/074820

§ 371 Date: Feb. 22, 2022

(30) Foreign Application Priority Data
Sep. 5, 2019 (EP) 19195629.1

(51) Int. Cl. C12Q 1/6886; C12Q 1/25

(52) U.S. Cl. CPC C12Q 1/6886; C12Q 1/25

(57) ABSTRACT

The present invention relates to the treatment of AML. The inventors previously discovered a new epigenetic biomarker in a cohort of CN-AML patients; this consists in a strong enrichment in the H3K27me3 histone mark located on a 70 Kb part of the major histone cluster 1 (HIST1) that separates patients into two distinguishable groups defined as H3K27me3HIST1^{low} and H3K27me3HIST1^{high}. Patients harboring the H3K27me3 HIST1 epigenetic mark had a better event free survival. This first observation suggests that H3K27me3HIST1^{high} patients may develop a less aggressive disease. Molecular characterisation of H3K27me3HIST1^{high} patients showed that the linker histone H1d, but not the other histone H1 subtypes, was down-regulated in the H3K27me3 HIST1high group of patients. H1d knockdown primed ATRA differentiation, as assessed on CD11b/CD11c markers, morphological and gene expression analyses. These results suggested that targeting H1d could help to reverse the adverse immature phenotype of the H3K27me3 HIST1low group into the more favourable one of the H3K27me3 HIST1^{high} group of patients and thus could be a good target in AML. Thus the invention relates to an H1d inhibitor for use in the treatment of acute myeloid leukemia (AML) in a patient in need thereof.

### FIELD OF THE INVENTION

The present invention relates an H1d inhibitor for use in the treatment of acute myeloid leukemia (AML) in a patient in need thereof.

### BACKGROUND OF THE INVENTION

Acute myeloid leukemias (AMLs) are a heterogeneous group of severe hematological malignancies that arise through the acquisition of oncogenic mutations by hematopoietic progenitor cells. As a consequence, AML differentiation program is variously impaired. Patient prognosis mainly depends on cytogenetics and molecular alterations. Cytogenetically normal (CN) AML patients are usually assigned to an intermediate prognosis group that can be further subdivided through the detection of mutations in a growing number of genes.1 Mutations in the nucleophosmin 1 (NPM1) gene are some of the commonest molecular lesions identified to date occurring in ≥50% of cases with CN-AML. NPM1 mutations result in the generation of a nuclear export signal causing the delocalization of the protein from the nucleoli to the cytoplasm.2 Analyses of large numbers of patients have shown that NPM1 mutations are associated with a relatively favorable prognosis that can be mitigated by two coexisting mutations frequently associated with NPM1mut: Internal tandem duplications (ITD) of the tyrosine kinase 3 (FLT3) and DNA-methyl transferase 3A (DNMT3A). In their absence, NPM1mut CN-AMLs have a relatively favorable prognosis, whereas FLT3-ITD mutation and/or mutation in DNA-methyl transferase 3A (DNMT3A) predict an increased risk of relapse and poorer outcome.3-5

Recent reports shed light on the importance of dysregulated epigenetic mechanisms in AML pathogenesis.6 Enhancer of zeste homolog 2 (EZH2) is a histone lysine methyl transferase (KMT) that belongs to the PRC2 complex. EZH2 catalyzes di- and tri-methylation of histone H3 lysine 27 (H3K27), resulting in transcriptional repression. Deregulation of EZH2 is strongly oncogenic but its role in hematological malignancies varies depending on the cellular context.? Gain-of function mutations are frequently found in germinal-center B-cell lymphomas8 while loss of function mutations are associated with 18% of T-acute lymphoblastic leukemias9 and 3-13% of myelodysplastic or myeloproliferative syndromes with a worse outcome.10 EZH2 mutation is rare in AML (≈2%)4, but the breadth of epigenetic deregulations includes far more than the consequences of somatic mutations in epigenetic modifiers3. Indeed, other mechanisms such as protein degradation can lead to diminished EZH2 expression and AML blasts resistance to chemotherapy.11. Furthermore, some AML display abnormal genome-wide DNA methylation patterns in the absence of mutations in known epigenetic modifiers.12,13

### SUMMARY OF THE INVENTION

The inventors previously discovered a new epigenetic biomarker in a cohort of CN-AML patients; this consists in a strong enrichment in the H3K27me3 histone mark located on a 70 Kb part of the major histone cluster 1 (HIST1) that separates patients into two distinguishable groups defined as H3K27me3HIST1^{low} and H3K27me3HIST1^{high} (see patent application WO2015169906). They observed a clear association between H3K27me3 HIST1 epigenetic mark and the presence of NPM1 mutations. Patients harboring the H3K27me3 HIST1 epigenetic mark had a better event free survival. This first observation suggests that H3K27me3HIST1^{high} patients may develop a less aggressive disease. Molecular characterisation of H3K27me3HIST1^{high} patients showed that the linker histone H1d, but not the other histone H1 subtypes, was down-regulated in the H3K27me3 HIST1^{high} group of patients. H1d knockdown primed ATRA differentiation, as assessed on CD11b/CD11c markers, morphological and gene expression analyses. These results suggested that targeting H1d could help to reverse the adverse immature phenotype of the H3K27me3 HIST1^{low} group into the more favourable one of the H3K27me3 HIST1^{high} group of patients and thus could be a good target in AML.

Thus, the present invention relates an H1d inhibitor for use in the treatment of acute myeloid leukemia (AML) in a patient in need thereof. Particularly, the invention is defined by its claims.

### DETAILED DESCRIPTION OF THE INVENTION

The invention refers to an H1d inhibitor for use in the treatment of acute myeloid leukemia (AML) in a patient in need thereof.

An H1d inhibitor could be very suitable to sensibilize cancerous cells to therapeutic compounds used to treat AML. These therapeutic compounds already used to treat AML are for example all-trans retinoic acid (ATRA; tretinoin), gemtuzumab ozogamicin, the combination of methotrexate, mercaptopurine and ATRA, demethylating agent, or chemotherapy such as cytarabine (araC), docetaxel, etoposide, idarubicin, volasertib, tozasertib (VX-680), nutlin 3 or olaparib. Allograft can also be used to treat AML.

Compounds useful for the treatment of AML are well known in the art (see for example Sweet K. et al., 2014).

As used herein, the term “chemotherapy” refers to use of chemotherapeutic agents to treat a subject. As used herein, the term “chemotherapeutic agent” or “anti-cancer agents” refers to chemical compounds that are effective in inhibiting tumor growth.

Thus the invention also relates to an H1d inhibitor to sensibilize cancerous cells to therapeutic compounds used to treat AML.

In other words, the invention relates to an H1d inhibitor for use to sensibilize cancerous cells to therapeutic compounds used to treat AML

In other words, the invention relates to an H1d inhibitor to sensibilize AML cancerous cells to therapeutic compounds used to treat AML.

In another particular embodiment, the invention relates to an i) H1d inhibitor and a ii) therapeutic compound used to treat AML or allograft according to the invention as a combined preparation for simultaneous, separate or sequential use in the treatment of AML or use in the sensilization of AML cancerous cells.

In some embodiment, the therapeutic compounds used to treat AML are chemotherapeutic agents.

In some embodiment, the therapeutic compounds used to treat AML is selected from cytarabine (araC), volasertib, tozasertib (VX-680), nutlin 3 or olaparib.

As used herein, the term “H1d” also known as histone H1.3 is a protein that in humans is encoded by the HIST1H1D gene. Histones are basic nuclear proteins responsible for nucleosome structure of the chromosomal fiber in eukaryotes. Two molecules of each of the four core histones (H2A, H2B, H3, and H4) form an octamer, around which approximately 146 bp of DNA is wrapped in repeating units, called nucleosomes. The linker histone, H1, interacts with linker DNA between nucleosomes and functions in the compaction of chromatin into higher order structures. This gene is intronless and encodes a member of the histone H1 family. The Entrez Gene ID number is 3007 and the Uniprot accession number is P16402).

According to the invention the AML can be a Cytogenetically normal AML (CN-AML), an acute promyelocytic leukemia (APL) an acute myeloid leukemia with trisomy 8 or an acute leukemia with MLL translocations.

In a particular embodiment, the invention also relates to an H1d inhibitor for use in the treatment of acute myeloid leukemia (AML) with NPM1 mutations in a patient in need thereof.

As used herein, the term “patient” denotes a mammal, such as a rodent, a feline, a canine, and a primate. Particularly, the patient according to the invention is a human.

In some embodiment, the patient is suffering from CN-AML.

In some embodiment, the patient is suffering from CN-AML and has a NPM1 mutations (“NMP1-mut CN-AML patient”)

In some embodiment, the patient is H3K27me3 HIST1^{high} patient suffering from CN-AML.

In some embodiment, the patient is H3K27me3 HIST1^{low} patient suffering from CN-AML.

In some embodiment, the patient is H3K27me3 HIST1^{high} patient suffering from CN-AML and having a NPM1 mutations.

In some embodiment, the patient is H3K27me3 HIST1^{low} patient suffering from CN-AML and having a NPM1 mutations.

As used herein, the term “H3K27me3 HIST1^{high} patient” has it general meaning in the art refers to patient exhibiting a high level of tri-methylated H3K27 in the HIST1 cluster. The patient harbours a strong enrichment in the H3K27me3 histone mark located on a 70 Kb part of the major histone cluster 1 (HIST1). H3K27me3 HIST1^{high} patient are defined in the patent application WO2015169906.

As used herein, the term “H3K27me3 HIST1^{low} patient” has it general meaning in the art refers to patient exhibiting a low level of tri-methylated H3K27 in the HIST1 cluster.

As used herein, the term “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The term “H1d inhibitor” denotes molecules or compound which can inhibit the activity of the histone (e.g. inhibit the interaction of the histone with linker DNA between nucleosomes and functions in the compaction of chromatin into higher order structures.) or a molecule or compound which destabilizes the histone.

The term “H1d inhibitor” also denotes inhibitors of the expression of the gene (HIST1H1D) coding for the protein.

In one embodiment, the inhibitors according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In one embodiment, the inhibitor according to the invention (inhibitor of Hid) is an antibody. Antibodies directed against H1d can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against H1d can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-H1d single chain antibodies. Compounds useful in practicing the present invention also include anti-H1d antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to H1d.

Humanized anti-H1d antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of H1d are selected.

In a particular embodiment, the anti-H1d antibody according to the invention may be the ab24174antibody as send by Abcam.

In another embodiment, the antibody according to the invention is a single domain antibody against H1d. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of H1d are selected.

In one embodiment, the compound according to the invention is a polypeptide.

In a particular embodiment the polypeptide is an antagonist of H1d and is capable to prevent the function of H1d. Particularly, the polypeptide can be a mutated H1d protein or a similar protein without the function of H1d.

In one embodiment, the polypeptide of the invention may be linked to a cell-penetrating peptide” to allow the penetration of the polypeptide in the cell.

The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E. coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the H1d inhibitor according to the invention is an inhibitor of H1d gene expression.

Small inhibitory RNAs (siRNAs) can also function as inhibitors of H1D expression for use in the present invention. H1D gene expression can be reduced by contacting a patient or cell with a small double stranded RNA (dsRNA), or a vector or construct causing the production of a small double stranded RNA, such that H1d gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of H1d gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of H1d mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of H1d gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing H1d. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In another embodiment, the invention relates to a method for treating AML comprising administering to a patient in need thereof a therapeutically effective amount of an inhibitor of H1d.

In a particular embodiment, an endonuclease can be used to reduce or abolish the expression of the gene, transcript or protein variants of ERFE.

Indeed, as an alternative to more conventional approaches, such as cDNA overexpression or downregulation by RNA interference, new technologies provide the means to manipulate the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error prone non homologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In a particular embodiment, a CRISPR-cas9 can be used to apply the tri-methylation on H3K27 and thus inhibits the expression of H1d. Particularly, the dCas9-EZH2 (which will express the histone methyltransferase Ezh2, the enzyme responsible of the tri-methylation) can be used to apply the tri-methylation on H3K27 and thus inhibits the expression of H1d (see for example O'Geen H, Ren C, Nicolet C M, Perez A A, Halmai J, Le V M, Mackay J P, Farnham P J, Segal D J (2017) dCas9-based epigenome editing suggests acquisition of histone methylation is not sufficient for target gene repression. Nucleic Acids Res 45: 9901-9916).

In order to test the functionality of a putative H1d inhibitor a test is necessary. For that purpose, to identify H1d inhibitors, we can evaluate the expression level of the gene (by PCR for example) in presence or not of the putative inhibitor. With an inhibitor of H1d, the expression level of the gene H1d will be diminished.

### Therapeutic Composition

Another object of the invention relates to a therapeutic composition comprising an inhibitor of H1d according to the invention for use in the treatment of AML, in a patient in need thereof.

In another particular embodiment, the invention relates to a therapeutic composition comprising an inhibitor of H1d according to the invention to sensibilize cancerous cells to therapeutic compounds used to treat AML.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising an agonist, antagonist or inhibitor of the expression according to the invention and a further therapeutic active agent.

For example, anti-cancer agents may be added to the pharmaceutical composition as described below.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine (AraC), anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan, estramustine, etoposide, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, idrarubucin, volasertib, tozasertib (VX-680), nutlin 3, olaparib and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating AML, the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiri de, tetrahydrocannabinols, thiefhylperazine, thioproperazine and tropisetron. In a preferred embodiment, the antiemetic agent is granisetron or ondansetron.

In another embodiment, the further therapeutic active agent can be an hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

### Predictive Method

Another object of the invention relates to an in vitro method for predicting the survival time of a patient suffering from acute myeloid leukemia (AML) comprising: i) determining, in a sample obtained from the patient, the expression level of at least one gene selected in the group consisting in CYBB, FCN1, CLEC4 and ITGAM; ii) comparing the expression level of the genes determined at step i) with their predetermined reference values and iii) providing a bad prognosis when the expression levels determined at step i) are higher than their predetermined reference values, or providing a good prognosis when the expression level determined at step i) are lower than their predetermined reference values.

In a particular embodiment, the expression level of 2, 3 or the 4 genes is obtained.

According to the invention, the acute myeloid leukemia (AML) can be an acute myeloid leukemia with cytogenetically normal AML (CT-AML), an acute promyelocytic leukemia (APL) an acute myeloid leukemia with trisomy 8 or an acute leukemia with MLL translocation.

According to the invention the patient with a bad prognostic can be treated with anti-AML compound like demethylating agent, by allograft or with an H1d inhibitor according to the invention alone or in combination with all-trans retinoic acid (ATRA; tretinoin), gemtuzumab ozogamicin or the combination of methotrexate, mercaptopurine and ATRA.

As used herein the term “allograft” denotes a patient who has been treated by hematopoietic stem cell transplantation (HSCT). According to the term allograft, hematopoietic stem cells come from a donor related or not to the recipient but of the same species.

In another embodiment, methods according to the invention may be useful for predicting the overall survival (OS) of a patient suffering from acute myeloid leukemia (AML) or for predicting the free survival (FS) of a patient suffering from acute myeloid leukemia (AML).

In a particular embodiment, the invention relates to a method for predicting the overall survival (OS) of a patient suffering from acute myeloid leukemia (AML) comprising: i) determining, in a sample obtained from the patient, the expression level of at least one gene selected in the group consisting in CYBB, FCN1, CLEC4 and ITGAM; ii) comparing the expression level of the genes determined at step i) with their predetermined reference values and iii) providing a bad prognosis when the expression levels determined at step i) are higher than their predetermined reference values, or providing a good prognosis when the expression level determined at step i) are lower than their predetermined reference values.

In a particular embodiment, the invention relates to a method for predicting the free survival (FS) of a patient suffering from acute myeloid leukemia (AML) comprising: i) determining, in a sample obtained from the patient, the expression level of at least one gene selected in the group consisting in CYBB, FCN1, CLEC4 and ITGAM; ii) comparing the expression level of the genes determined at step i) with their predetermined reference values and iii) providing a bad prognosis when the expression levels determined at step i) are higher than their predetermined reference values, or providing a good prognosis when the expression level determined at step i) are lower than their predetermined reference values.

As used herein, the term “Overall survival (OS)” denotes the percentage of people in a study or treatment group who are still alive for a certain period of time after they were diagnosed with or started treatment for a disease, such as AML (according to the invention). The overall survival rate is often stated as a five-year survival rate, which is the percentage of people in a study or treatment group who are alive five years after their diagnosis or the start of treatment.

As used herein, the term “Free Survival (FS)” (or Event-Free-Survival) denotes the length of time after primary treatment for a cancer ends that the patient remains free of certain complications or events that the treatment was intended to prevent or delay. These events may include the return of the cancer or the onset of certain symptoms, such as bone pain from cancer that has spread to the bone.

As used herein, the term “CYBB” denotes the gene coding for the NADPH oxidase 2 (Nox2), also known as cytochrome b(558) subunit beta or Cytochrome b-245 heavy chain. The Entrez reference number is 1536.

As used herein, the term “FCN1” denotes the gene coding for the protein M-ficolin. The Entrez reference number is 2219.

As used herein, the term “CLEC4” denotes the gene coding for the protein C-type lectin domain family 4 member A. The Entrez reference number is 50856.

As used herein, the term “ITGAM” denotes the gene coding for the protein Integrin alpha M (ITGAM), one of the protein subunit that forms the heterodimeric integrin alpha-M beta-2 (αMβ2) molecule, also known as macrophage-1 antigen (Mac-1) or complement receptor 3 (CR3). The Entrez reference number is 3684.

As used herein the term “biological sample” in the context of the present invention is a biological sample isolated from a patient and can include, by way of example and not limitation, bodily fluids and/or tissue extracts such as homogenates or solubilized tissue obtained from a patient. Tissue extracts are obtained routinely from tissue biopsy and autopsy material. Bodily fluids useful in the present invention include blood, bone marrow aspirate, urine, saliva or any other bodily secretion or derivative thereof. As used herein “blood” includes whole blood, plasma, serum, circulating cells, constituents, or any derivative of blood. In a particular embodiment, the biological sample is a blood sample, more particularly a biological sample comprising circulating white blood cells (WBC).

Such samples include, but are not limited to, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample”.

In a particular embodiment, the sample includes nucleic acids.

Measuring the expression level of the genes listed above can be done by measuring the gene expression level of these genes or by measuring the level of the protein of the corresponding genes and can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook-A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[³ vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6diclllorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAIVIRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6, 130, 101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlsbad, Calif.).

Additional labels include, for example, radioisotopes (such as 3H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and passing the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test patient, optionally first passed by a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In another embodiment, the expression level is determined by metabolic imaging (see for example Yamashita T et al., Hepatology 2014, 60:1674-1685 or Ueno A et al., Journal of hepatology 2014, 61:1080-1087).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes such as the actin gene ACTB, ribosomal 18S gene, GUSB, PGK1, TFRC, GAPDH, GUSB, TBP and ABL1. This normalization allows the comparison of the expression level in one sample, e.g., a patient sample, to another sample, or between samples from different sources.

According to the invention, the level of the proteins of the genes listed above may also be measured and can be performed by a variety of techniques well known in the art. For measuring these proteins, techniques like ELISA (see below) allowing to measure the level of the soluble proteins are particularly suitable.

In the present application, the “level of protein” or the “protein level expression” or the “protein concentration” means the quantity or concentration of said protein. In another embodiment, the “level of protein” means the level of the proteins fragments. In still another embodiment, the “level of protein” means the quantitative measurement of the proteins expression relative to a negative control.

According to the invention, the protein level of the proteins may be measured at the surface of the tumor cells or in an extracellular context (for example in blood or plasma).

Typically protein concentration may be measured for example by capillary electrophoresis-mass spectroscopy technique (CE-MS) or ELISA performed on the sample.

Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS) etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports, which can be used in the practice of the invention, include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Methods of the invention may comprise a step consisting of comparing the proteins and fragments concentration in circulating cells with a control value. As used herein, “concentration of protein” refers to an amount or a concentration of a transcription product, for instance the proteins. Typically, a level of a protein can be expressed as nanograms per microgram of tissue or nanograms per milliliter of a culture medium, for example. Alternatively, relative units can be employed to describe a concentration. In a particular embodiment, “concentration of proteins” may refer to fragments of the proteins. Thus, in a particular embodiment, fragment of the proteins may also be measured.

Predetermined reference values used for comparison may comprise “cut-off” or “threshold” values that may be determined as described herein. Each reference (“cut-off”) value for the genes' expression may be predetermined by carrying out a method comprising the steps of

a) providing a collection of samples from patients suffering of AML (after diagnosis of AML for example);

b) determining the expression level of the genes or of the corresponding proteins for each sample contained in the collection provided at step a);

c) ranking the tumor tissue samples according to said gene or protein expression level and determining a threshold value above which the expression level is said to be “high” and below which the expression level is said to be “low”;

d) quantitatively defining the threshold/cut-off/reference value by determining the number of copies of the said gene/protein corresponding to the threshold/cut-off/reference value; to be done by constructing a calibration curve using known input quantities of cDNA or protein for the said gene;

e) classifying said samples in pairs of subsets of increasing, respectively decreasing, number of members ranked according to their expression level,

f) providing, for each sample provided at step a), information relating to the actual clinical outcome for the corresponding cancer patient (i.e. the duration of the overall survival (OS));

g) for each pair of subsets of samples, obtaining a Kaplan Meier percentage of survival curve;

h) for each pair of subsets of samples calculating the statistical significance (p value) between both subsets

i) selecting as reference value for the expression level, the value of expression level for which the p value is the smallest.

For example the expression level of the genes or proteins has been assessed for 100 AML samples from 100 patients. The 100 samples are ranked according to their expression level. Sample 1 has the highest expression level and sample 100 has the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding AML patient, Kaplan Meier curves are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels.

In routine work, the reference value (cut-off value) may be used in the present method to discriminate AML samples and therefore the corresponding patients.

Kaplan-Meier curves of percentage of survival as a function of time are commonly used to measure the fraction of patients living for a certain amount of time after treatment and are well known by the person skilled in the art.

The man skilled in the art also understands that the same technique of assessment of the expression level of a gene should of course be used for obtaining the reference value and thereafter for assessment of the expression level of a gene of a patient patented to the method of the invention.

Such predetermined reference values of expression level may be determined for any gene defined above.

In a further embodiment of the invention, methods of the invention comprise measuring the expression level of the genes according to the invention with at least one further biomarker or prognostic score.

The term “biomarker”, as used herein, refers generally to a cytogenetic marker, a molecule, the expression of which in a sample from a patient can be detected by standard methods in the art (as well as those disclosed herein), and is predictive or denotes a condition of the patient from which it was obtained.

Various validated prognostic biomarkers or prognostic scores may be combined to the measuring of the expression level of the genes according to the invention in order to improve methods of the invention and especially some parameters such as the specificity (see for example Cornelissen et al. 2012).

For example, the other biomarkers may be selected from the group of AML biomarkers consisting of cytogenetics markers (like t(8;21), t(15;17), inv(16), t(16;16), t(9;11),-5, -7, 5q-, 7q-, 11q23, excl. t(9;11), Inv(3), t(3;3), t(6;9), t(9;22) see for example Grimwade et al., 2010 or Byrd et al., 2002), lactate dehydrogenase (see for example Haferlach et al 2003), FLT3, NPM1, CEBPα (see for example Schnittger et al., 2002).

The prognostic scores that may be combined to the method of the invention may be for example the Hematopoietic Cell Transplantation Comorbidity Index (HCT-CI) (Sorror et al 2005), the comorbidity and disease status (Sorror et al 2007) or the disease risk index (DRI) (Armand et al 2012).

According to the invention, detection of a mutation in the gene NPM1 can be added to the determination of the expression level of the genes of the invention for predicting the survival time of a patient suffering from acute myeloid leukemia (AML).

As used herein, the term “NPM1” denotes a gene coding fort the protein nucleophosmin (NPM), also known as nucleolar phosphoprotein B23 or numatrin. The protein NPM1 is associated with nucleolar ribonucleoprotein structures and bind single-stranded and double-stranded nucleic acids, but it binds preferentially G-Quadruplex forming nucleic acids. NPM1 mutations are known to be biomarkers for AML (Falini B et al., 2009).

Thus, the invention also relates to an in vitro method for predicting the survival time of a patient suffering from acute myeloid leukemia (AML) comprising determining, in a biological sample from the patient the expression level of the gene according to the invention and if a mutation in the gene NPM1 is present.

According to the invention, determination of the level expression for genes of the HIST1 cluster can be added to the determination of the expression level of the genes of the invention for predicting the survival time of a patient suffering from acute myeloid leukemia (AML).

According to the invention, the genes of the HIST1 cluster can be HIST1H2BG, HIST1H2AE, HIST1H3E, HIST1H1D, HIST1H4F, HIST1H4G, HIST1H3F, HIST1H2BH, HIST1H3G, HIST1H2BI or HIST1H4H.

Accession numbers of the different genes are: HIST1H2BG: Ref Seq NM_003518.3 GenBank: M60750.1; HIST1H2AE: Ref Seq NM_021052 GenBank: M60752; HIST1H3E: Ref Seq NM_003532 GenBank: M60746; HIST1H1D: Ref Seq NM_005320 GeneBank: M60747; HIST1H4F: Ref Seq NM_003540 GeneBank: M60749; HIST1H4G: Ref Seq NM_003547 GeneBank: Z80788; HIST1H3F: Ref Seq NM_021018 GeneBank: Z80786; HIST1H2BH: Ref Seq NM_003524 GeneBank: Z80781; HIST1H3G: Ref Seq NM_003534 GeneBank: Z80785 and HIST1H2BI: Ref Seq NM_003525 GeneBank: Z80782.

Thus, the invention also relates to an in vitro method for predicting the survival time of a patient suffering from acute myeloid leukemia (AML) comprising determining, in a biological sample from the patient, the expression level of the genes of the invention and the expression level of at least one gene selected in the group consisting of HIST1H2BG, HIST1H2AE, HIST1H3E, HIST1H1D, HIST1H4F, HIST1H4G, HIST1H3F, HIST1H2BH, HIST1H3G, HIST1H2BI or HIST1H4H.

According to the invention, determination of the epigenetic profile of the H3K27 (see patent application WO2015169906) can be added to the determination of the expression level of the genes of the invention for predicting the survival time of a patient suffering from acute myeloid leukemia (AML).

More particularly, the epigenetic profile of the H3K27 at the HIST1 cluster located on 6p22.2 can be determined.

In a particular embodiment, the invention relates to an in vitro method for predicting the survival time of a subject suffering from acute myeloid leukemia (AML) comprising i) determining in a sample obtained from the subject the histone methylation profile level of H3K27 at the HIST1 cluster located on 6p22.2 and the expression level of at least one gene selected in the group consisting in CYBB, FCN1, CLEC4 and ITGAM ii) comparing the histone methylation profile level of H3K27 at the HIST1 cluster located on 6p22.2 at step i) with its predetermined reference value and comparing the expression level of the genes determined at step i) with their predetermined reference values and iii) providing a good prognosis when the histone methylation profile level determined at step i) is higher than its predetermined reference value and when the expression level determined at step i) are lower than their predetermined reference values, or providing a bad prognosis when the histone methylation profile level determined at step i) is lower than its predetermined reference value and when the expression levels determined at step i) are higher than their predetermined reference values.

The present invention also relates to kits for predicting the survival time of a patient suffering from acute myeloid leukemia (AML) comprising means for determining, in a biological sample from the patient the expression level of the gene of the invention.

The invention also refers to a method of treatment of an AML in a patient in need thereof comprising the step of:
1. determining if the patient as a good or a bad prognosis according to the invention and;
2. administrating to said patient a compound useful for the treatment of AML, as defined in the present invention when the prognosis of the patient is bad as determined by methods of the invention.

The treatment used can be an allograft (allogeneic stem cell transplantation) or all compound used to treat AML like all-trans retinoic acid (ATRA; tretinoin), gemtuzumab ozogamicin, the combination of methotrexate, mercaptopurine and ATRA or demethylating agent and others anti-cancer agents.

The inventors also determined the response to chemotherapy according to H3K27me3 status and demonstrated that H3K27me3HIST1^{high} patients were more sensitive to chemotherapy.

Thus, in another aspect, the invention refers to an in vitro method for predicting chemotherapeutic agent response of a patient suffering from AML in need thereof, comprising i) determining in a sample obtained from the subject the histone tri-methylation profile level of H3K27 ii) comparing the histone tri-methylation profile level of H3K27 at step i) with its predetermined reference value and iii) concluding that the patient will respond to chemotherapeutic agent when the histone tri-methylation profile level determined at step i) is higher than its predetermined reference value, or concluding that the patient will not respond to chemotherapeutic agent when the histone tri-methylation profile level determined at step i) is lower than its predetermined reference value.

In some embodiment, the patient is suffering from CN-AML.

In some embodiment, the patient is suffering from CN-AML and has a NPM1 mutations (“NMP1-mut CN-AML patient”)

In some embodiment, the chemotherapeutic agent is selected from cytarabine (araC), volasertib, tozasertib (VX-680), nutlin 3 or olaparib.

As used herein, the term “histone methylation profile level of H3K27” denotes the level of methylation of the Histone H3 on the lysine 27 in the HIST1 cluster located on 6p22.2 (26216000-2628500) that is to say the number of CH3 group on the Histone H3 on the lysine 27.

The present invention also relates to kits for predicting chemotherapeutic agent response of a patient suffering from acute myeloid leukemia (AML) comprising means for determining, in a biological sample from the patient the expression level of the histone tri-methylation profile level of H3K27.

The invention also refers to a method of treatment of an AML in a patient in need thereof comprising the step of:
1. determining if the patient is a good or bad responder to chemotherapeutic agent according to the invention and;
2. administrating to said patient a compound useful for the treatment of AML as defined in the present invention when the patient is a good responder to chemotherapeutic agent as determined by methods of the invention.

The invention will be further illustrated by the following figures and examples. However, these examples and figures should not be interpreted in any way as limiting the scope of the present invention.

### FIGURES

FIG. 1: H3K27me3 HIST1^{high} is associated with a myelomonocytic GEP

(A) Expression of three genes associated with granulocytic functions according to H3K27me3 HIST1 status. CYBB (Cytochrome B-245 Beta Chain); FCN1 (Ficolin 1); CLEC4A (C-Type Lectin Domain Family 4 Member A). Data are represented in relative expression to HPRT (B) Expression analysis of patients from the TGCA and GSE 61804 cohorts separated according to the 3-HIST1 mRNA signature as described in the text (B) myelomonocytic genes TNFSF10 (TNF Superfamily Member 10), FCN1 (Ficolin 1), CLEC4A (C-Type Lectin Domain Family 4 Member A), ITGAM (Integrin Subunit Alpha M) were analyzed in 3-HIST1 mRNA^{low} (N=114) and in 3-HIST1 mRNA^{high} (N=79) patient samples.

FIG. 2: H1d KD promotes granulocytic differentiation in ATRA-treated OCI-AML3 cell line

(A) OCI-AML cells were stably infected by a doxycycline inducible shCtrl or shH1d. Expression of the main histone H1 genes (HIST1H1B, HIST1H1C, HIST1H1D, HIST1H1E, HIST1H1F0 and HIST1HIFx) were analyzed by qPCR in shCtrl and sh #1 without (Dox−) or with 6-days-induction of doxycycline (Dox+). Data represent three independent Dox inductions. Results are normalized on HPRT and expressed in fold change (FC) between Doxycyline treated (Dox+) and non-treated conditions (Dox−). (B) Percentage of CD11b positive cells in shCtrl and sh #1 upon or not (Dox−) 6-days-induction with 2 μg/mL doxycycline (Dox+), without ATRA (wo ATRA) or with 72 hours of ATRA treatment (0.5 μM or 1 μM). (C) Percentage of CD11b-CD11c cells upon 72 h of ATRA-treatment (0.5 μM) in shCtrl and sh #1 conditions. Data represent three independent experiments. Statistical significance was estimated using Mann Whitney test * p<0.05; **p<0.005. (D) Cytological analysis of shCtrl and sh #1 clones upon or not (Dox−) 6-days-induction with doxycycline (Dox+) and after 96 h of ATRA treatment (0.5 μM). (D) May-Grunwald Giemsa coloration of shCtrl and sh #1 clones after 6-days-induction with doxycycline and 96 h hours of ATRA treatment. Black arrows are pointing cytoplasmic azurophilic granules. (E) Expression analysis of two genes associated with ATRA-induced differentiation: CYBB (Cytochrome B-245 Beta Chain) and ITGAM (Integrin Subunit Alpha M) in untreated OCI-AML3 and in ATRA-treated (1 μM) sh #1 without or with doxycycline induction. Data represent three independent experiments. Gene expression was normalized to two housekeeping genes (PGK1 and PPIA). Statistical significance was estimated using T-test (* one-tailed p value <0.05).

FIG. 3: Chemogram on H3K27me3HIST1^{high} and H3K27me3HIST1^{low} AML cells. (A) H3K27me3HIST1^{high} and H3K27me3HIST1^{low} AML cells were seeded in 96 well plates to test cell viability after drug treatment (B) 78 drugs have been tested, among witch 14 presented a fold-change >5 between H3K27me3HIST1^{high} and H3K27me3HIST1^{low} patients. 4 drugs showed a significant difference (p value>0,05; unpaired Two-tailed t test). (C) IC50 of Chemo drugs and (D) the 4 drugs with significant difference between H3K27me3HIST1^{high} and H3K27me3HIST1^{low} patients. Unpaired Two-tailed t test was performed. NS non-significant; *p<0,05; ** p<0,01; *** p<0,001.

FIG. 4: Detection of leukemic initiating cells after ARAC treatment. (A) H3K27me3HIST1^{high} and H3K27me3HIST1^{low} AML secondary transplants were injected in immunocompromised mice (NSG). At disease detection, mice were treated with a first round of ARAC (T1). Blasts in the peripheral blood were followed and a second ARAC treatment (T2) was administrated. (B) Frequency of leukemic initiating cells were analyzed in the BM of the transplanted mice. Percentages are calculated in relation to CD45 and CD33 positive cells detected.

### EXAMPLE

Material & Methods

Patient Samples.

This study was performed after approval by our institutional review board. Patient's samples were obtained after informed consent in accordance with the Declaration of Helsinki and stored at Institut Paoli-Calmettes/Centre de Recherche en Cancérologie de Marseille (IPC/CRCM) Tumor Bank and in the Groupe Ouest Est d'Etude des Leucémies Aiguës et autres Maladies du Sang (GOELAMS) repository.

Histone Gene Nomenclature.

The large cluster of histone gene HIST1 on human chromosome 6p22 is 2.1 Mb large and contains 55 histone genes. Five genes encode the canonical somatic histone linkers H1 (HIST1H1.A-E) while sets of 10-20 genes encode each of the core histone proteins (H2A, H2B, H3 and H4). Two non-canonical somatic histone linker genes, H1.0 and H1X are encoded outside the HIST1 cluster on 22q13 and 3q21, respectively. Each of these genes is translated into a unique mRNA with a distinct 5′ and 3′ and promoter, as well as slight nucleotide changes in the coding region. These genes are named according to their location in the cluster.14

ChIP-qPCR.

ChIP was performed as previously described.15 Quantification of ChIPed DNA was performed by real-time PCR using the SsoADV Univer SYBR Green Supermix (Biorad) and detected with a CFX96 Real-Time PCR Detection System (Biorad). IgG control “cycle over the threshold” Ct values were subtracted to Input or IP Ct values and converted into bound value by 2(−(IP Ct or input Ct-IgG IP Ct)). Data are expressed as % of bound/input and double normalization was done as previously described.15

Gene Expression Profiling.

RNA expression profiling of NPM1mut CN-AML was done with Affymetrix Human gene ST 2.0 DNA microarrays (see Supplemental data).

Protein Analysis.

Cellular fractionation was carried out using the subcellular protein fractionation kit for cultured cells (Thermofisher). Mass spectrometry procedures are explained in supplemental data. Immunoblot were performed as previously described.16 Antibodies used were anti-Histone H1.3 (H1D) antibody (Abcam, ab24174, 1/750), anti-H1Antiboy (pAb) (Active Motif, 39707, 1/2000) and anti-H3 (Active Motif, cat 39163, 1/10000).

Cell Culture, shRNA Lentiviral Infection, Stable H1d Knockdown and Treatments.

The OCI-AML3 cells were grown in MEMα medium supplemented with 20% fetal bovine serum, 100 U/mL penicillin and 100 U/mL streptomycin at 37° C. in humidified atmosphere containing 5% CO2. H1d knockdown was achieved using doxycylin-induced Dharmacon™SMARTvector™ short hairpin RNA (n° V3SH7669-229784413). A non-silencing sh RNA (piSMART VSC10730) was used as a control (shCtrl). Cells containing the SMARTvector™ were selected on puromycin (2 μg/mL) during one day and sorted using ARIAIII cytometer before clonal selection. Three independent cellular clones (sh #1; sh #2; sh #3) were selected. KD of H1d protein was obtained by the addition on doxycycline (2 μg/mL) during 5-7 days. All-trans-retinoic acid (ATRA; Sigma) was resuspended at 10 mM in DMSO and stored at −20° C. Intermediate dilutions were made in culture medium before adding to the cells.

Flow Cytometry.

Flow cytometry analyses were performed using a BD-LSRII cytometer and analyzed using BD-DIVA Version 6.1.2 software (BD Biosciences). Antibodies used were CD11b-PE (Mac-1), 3:100, Beckman Coulter; CD11b-APC (M1/70), 1:500, eBioscience; CD11c-PeCy7 (BU-15), 3:100, Beckman Coulter; DRAQ7™, 1:400; Biostatus.

Morphological Analyses.

Cytospins were prepared by centrifugation in 200p1 PBS at a speed of 500 rpm for 5 min using Superfrost slides. Cytospin slides were stained at room temperature with May-Grunwald Giemsa (Sigma-Aldrich). 100 cells were counted in duplicate for each condition and examined for cellular morphology using an structured light ApoTome™ microscope (Zeiss, Munich, Germany) equipped with a 63× 1.4 plan ApoChromat objective and an Axiocam™ MRc5 camera.

Statistical Analyses.

Statistical analyses were carried out using R software (version 2.15.2) (The Comprehensive R Archive Network. http://www.cran.r-project.org/) and Graph Pad Prism (Graph Pad Software, San Diego, Calif., USA) and the significance of the differences between groups was determined via unpaired T-test, Mann-Whitney test or exact Fisher test. Data were presented as the median ±SEM. Overall survival (OS) and Leukemia-free survival (LFS) were calculated from the date of diagnosis to the date of death or to the date of relapse, death or last follows up, respectively. Follow-up was measured from the date of diagnosis to the date of last news for living patients. Survivals were calculated using the Kaplan-Meier method and were compared with the log-rank test. Uni- and multivariate survival analyses were done using Cox regression analysis (Wald test). Variables with a p-value <0.05 were tested in multivariate analysis. All statistical tests were two-sided at the 5% level of significance, unless where clearly indicated.

Results

H3K27Me3 Level on HIST1 Locus Association with Clinical and Molecular Features in CN-AML.

To further characterize the H3K27me3 HIST1 mark, we performed H3K27me3 ChIP-qPCR on samples obtained from 44 de novo CN-AML patients included in GOELAMS multicenter clinical trials LAM2006IR (NCT00860639) or LAM2007SA (NCT00590837). All patients received conventional induction chemotherapy and their characteristics are depicted in Table S 1. H3K27me3 level was determined at five HIST1 genomic locations as described previously.¹⁵ Heatmap showing H3K27me3 HIST1 gene enrichment of the GOELAMS patients confirmed the variation of H3K27me3 HIST1 level among CN-AML patients (data not shown). The mean of the 5 normalized H3K27me3 HIST1 values was calculated and this index showed a clear segregation of the H3K27me3 HIST1^{low} and H3K27me3 HIST1^{high} patients. With a cut off value at 15, approximately half of CN-AML patients displayed an H3K27me3 HIST1 enrichment mark (data not shown).

Concerning clinical and molecular features of patients, there was no association of H3K27me3 HIST1 status with age, gender, FLT3ITD, DNMT3A, IDH1, or CEBPA mutations. Nevertheless, we noted a strong association between NPM1 mutational status and H3K27me3 HIST1^{high} (data not shown) confirming our first observation.¹⁵ In addition we observed a significant association between H3K27me3 HIST1^{high} and the presence of IDH2 R140 mutation (64.3% versus 4.7%, P=0.01).

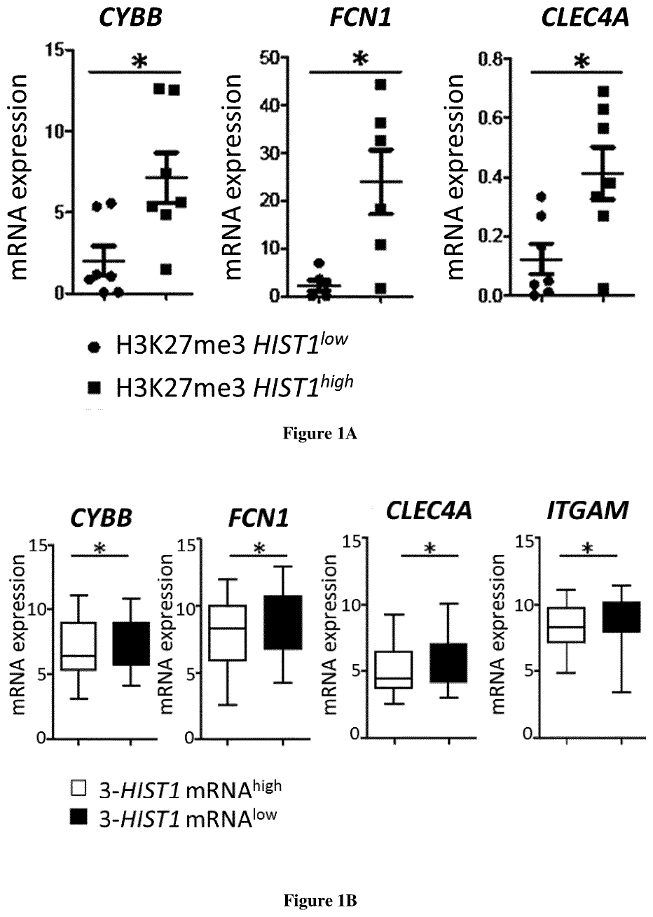

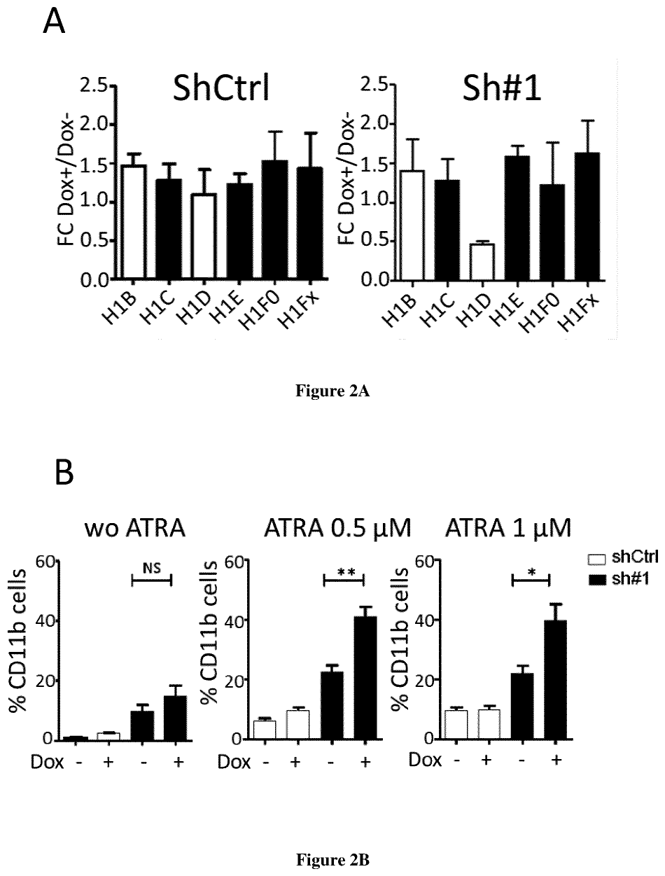

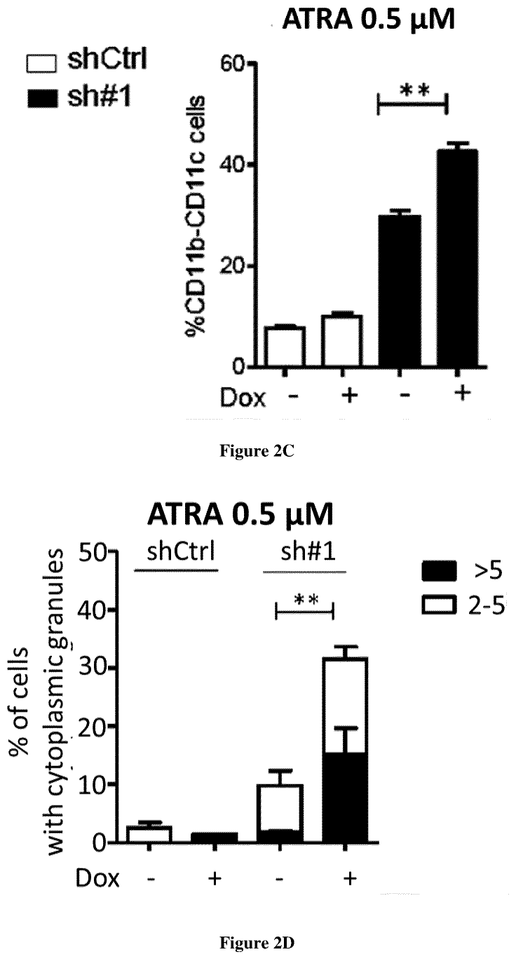

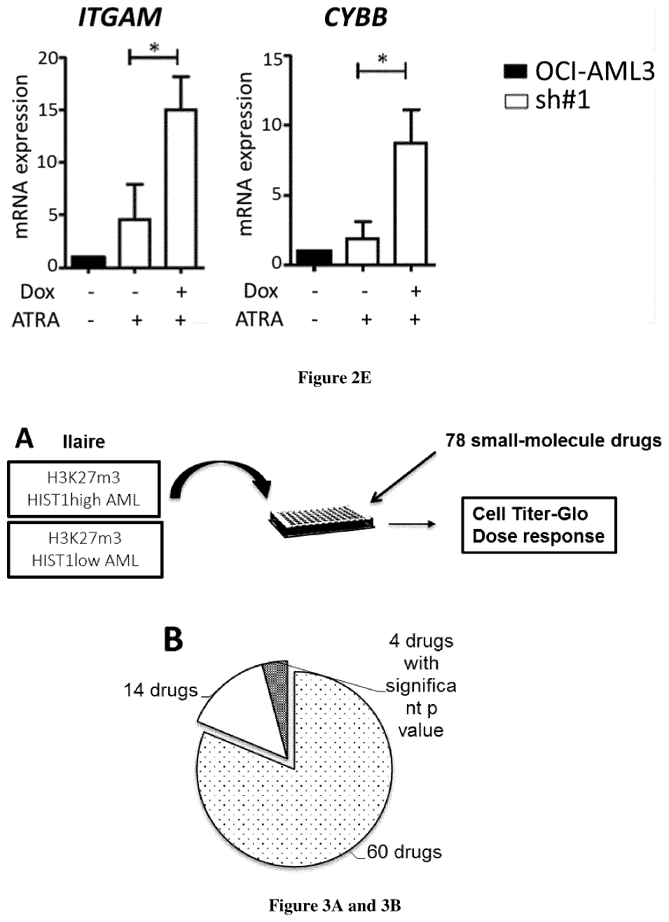

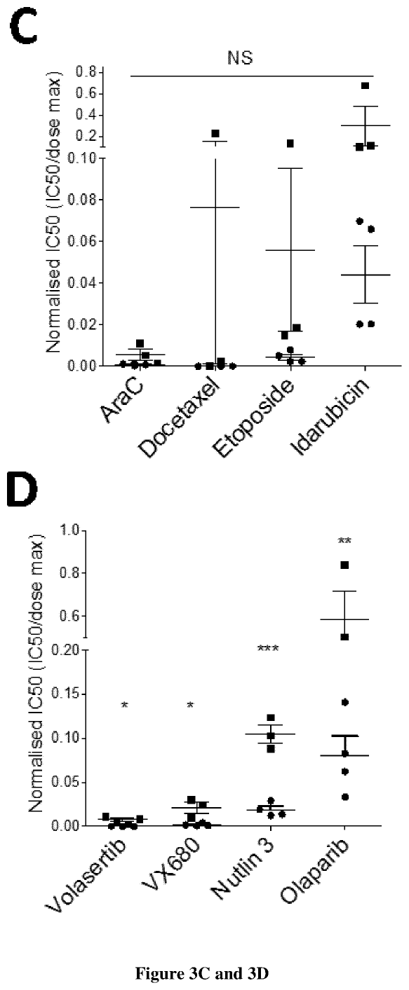

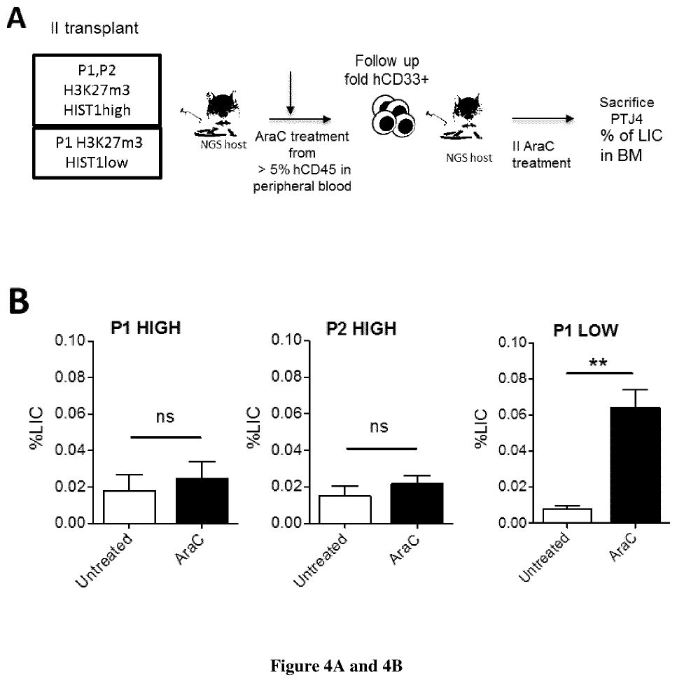

H3K27me3 HIST1^{high} patients had a significantly better OS and LFS as compared to H3K27me3 HIST1^{low} patients with a median OS of 42 months versus 14.6 months (HR, 2.5 [1.5-5.5]; P=0.04) and a median LFS of 21 months versus 9 months (HR, 2.5 [1.5-4.9]; P=0.02) (FIG. 1C). The survival gain was independent in multivariate analyses taking age and FLT3-ITD status into account (data not shown). Again, this observation confirms in an independent cohort the association of H3K27me3 HIST1^{high} with better prognosis that we previously reported.

Influence of H3K27Me3 HIST1 on Clinical Outcome of NPM1mut AML.

Given that NPM1 mutated AMLs represent a distinct entity in the World Health Organization (WHO) classification, commonly associated with a better risk prognosis⁵, we next analyzed the effect of the H3K27me3 HIST1 mark in the NPM1mut subgroup. We used the biological material obtained from NPM1mut patients, provided by the GOELAMS cell repository (n=33), and by the IPC tumor bank patients (these include 46 previously analyzed samples and 24 additional samples) (data not shown). Of these 103 NPM1mut CN-AML patients presented in figure S1, 75 (73%) were H3K27me3 HIST1^{high} (data not shown). In terms of molecular abnormalities, NPM1mut H3K27me3 HIST1^{high} group of patients was not enriched with DNMT3A or FLT3ITD mutations, two of the most frequently NPM1mut co-occurring alterations¹⁷. By contrast, IDH2 R140 was significantly overrepresented in the NPM1mut H3K27me3 HIST1^{high} subgroup in comparison with NPM1mut H3K27me3 HIST1^{low} (27.6% versus 7.6%, P=0.05). H3K27me3 HIST1^{high} leukemic cells had a significantly lower CD34 expression in comparison to their H3K27me3 HIST1^{low} counterparts (24.1% versus 71.4% CD34 positivity ≥2%, P=0.01) (Table 1).

In this group of NPM1mut-AMLs, OS and LFS were significantly better in H3K27me3 HIST1^{high} patients as compared to H3K27me3 HIST1^{low} patients, (median OS, 38.3 versus 15.7 months; HR, 2 [range, 1.0-3.0]; P=0.03; median LFS, 20.9 versus 10.6 months; HR, 2.7 [range, 1.3-5.7]; P=0.01) (FIG. 1D).

Influence of H3K27Me3 HIST1 on Clinical Outcome According to Age and Treatment Intensity.

H3K27me3 HIST1^{high} positive impact on survival was found both in young and older patients (>60 years) although more pronounced in older patients, probably due to the lesser proportion of allogeneic hematopoietic stem-cell transplantation HSCT in the older group of patients (11% vs 37,5%; P=0.002) (data not shown). HSCT represents a highly effective consolidation treatment proposed to patients with low comorbidities, according to their disease characteristics. In our series, 24 patients underwent HSCT (data not shown). As expected, the LFS difference was more apparent when censoring patients at HSCT (FIG. 1E). Moreover, H3K27me3 HIST1high favorable prognosis was predominant in the non-HSCT group of patients, contrary to the HSCT group of patients, suggesting that HSCT could salvage the H3K27me3 HIST1^{low} patient pejorative prognosis (data not shown).

H3K27Me3 HIST1 Impacts the NPM1mut/FLT3wt Patient Outcome.

Given that the outcome of NPM1mut disease treatment is influenced by co-occurring mutations (FLT3-ITD, DNMT3A)^{4,18}, we tested the influence of the H3K27me3 HIST1^{high} on survival depending on these mutations. As shown in FIG. 1E, H3K27me3 HIST1 status had a significant impact on survival in the FLT3wt/NPM1mut subgroup (n=53, median OS, 23.2 months versus 111.6 months; P=0.03; median LFS, 13.9 months versus 44.1 months; P=0.01, for H3K27me3 HIST1^{low} and H3K27me3 HIST1^{high}, respectively) but not in the FLT3ITD/NPM1mut subgroup (data not shown). No significant impact was found in the DNMT3wt/NPM1mut nor in the DNMT3Amut/NPM1mut subgroups in univariate analyses (data not shown). In multivariate analyses, the prognostic significance of H3K27me3 HIST1^{high} was independent of known clinical and molecular risk factors (data not shown).

All together our results suggest that H3K27me3 HIST1 status is an independent marker that could help to refine prognostic classification of NPM1mut CN-AML. This is particularly relevant in NPM1mut/FLT3wt patients.

Histone mRNA Gene Expression is Anti-Correlated to H3K27Me3 HIST1 Level and Predicts Patients Outcome of NPM1mut CN-AML Patients

To analyze the anti-correlation of histone mRNA level and the presence of the H3K27me3 mark, we selected three histone genes (HIST1H1D, HIST1H2BG and HIST1H1BH) spread over the H3K27me3 HIST1 islet and associated with clinical outcome in public data (see below) and measured their mRNA levels. Expression of these 3 genes was lower in H3K27me3 HIST1high patients (n=34) than in H3K27me3 HIST1low patients (n13) (data not shown).

We next asked whether expression of these genes, as a consequence of H3K27me3 repressive mark, was associated with patient survival. Given the small size of our cohort, we analyzed HIST1 gene expression in two published cohorts with publicly accessible clinical and mRNA expression data: TCGA3 and Metzeler17. NPM1mut CN-AML patients were identified by using a published gene expression signature that predicts the NPM1 mutational status 18 (see supplemental methods). Association of histone expression with survival was first tested for each of the 11 histone genes covered by the H3K27me3 HIST1 mark. This highlighted three histone genes, HIST1H1D HIST1H2BG and HIST1H2BH, for which high expression was associated with poor outcome (P=0.004, 0.015 and 0.044 respectively, data not shown). Then, we tested this 3-HIST1 mRNA signature in univariate analysis; 3-HIST1 mRNAlow patients had a favorable OS with a median OS of 17.7 months versus 9.6 months HR=1.66, Range, 1.13-2.42, P=0.009 (data not shown). Multivariate analyses showed that the 3-HIST1 mRNAlow status was associated with a better prognosis (HR=1.60, Range 1.60-2.31, P=0.01), independently of other markers including age, FAB classification and FLT3 status (Data not shown).

These results show that H3K27me3 HIST1^{high} is associated with a lower expression of histone genes, and that 3-HIST1 mRNAlow signature defines NPM1mut AML patients with better outcome.

GEP Associated with H3K27Me3 HIST1^{high} Identifies a “Mature Like” Phenotype.

We next characterized the gene expression profile (GEP) of H3K27me3 HIST1^{high} samples (n=16) in comparison to H3K27me3 HIST1^{low} samples (n=11) from the IPC cohort (data not shown). Eighty-one genes were differentially expressed (p<0.05, fold-change>1.5) between the two groups, 58 being up- and 23 being down-regulated in the H3K27me HIST1 high group (data not shown). Gene Set Enrichment Analysis (GSEA) identified upregulated genes in pathways active in myelomonocytic differentiation such as immune or inflammatory responses in H3K27me3 HIST1^{high} patients (data not shown). Down-regulated genes in these patients belong to cell cycle and chromatin regulation categories, including histone genes from the HIST1 cluster (data not shown). Using qPCR, we confirmed the higher expression of three genes involved in mature granulocyte functions, CYBB, FCN1 and CLEC4A²¹⁻²³ in H3K27me3 HIST1^{high} patients (FIG. 1A). These results are in line with the loss of CD34 observed in H3K27me3 HIST1^{high} patients (data not shown).

To further validate the relation between low mRNA level of HIST1 genes and the expression of granulocytic markers, we tested mRNA expression of myelomonocytic maturation genes (CYBB, FCN1, CLEC4 and ITGAM) in the TAGC and Metzeler cohorts of patients stratified with the previously defined 3-HIST1 mRNA signature. Results showed that the 3-HIST1 mRNA^{low} patient group over-expressed the differentiation genes in comparison to the 3-HIST1 mRNA^{high} group (FIG. 1B) corroborating our previous observation (FIG. 1A). Equally, genes such as SOCS2, CDK6, LAPTM4B and NGFRAP1—that were recently described as associated with a leukemic stem cell signature-²⁴ were less expressed in the 3-HIST1 mRNA^{low} patient group (data not shown).

Taken together, these results suggest that HIST1 mRNA down regulation by the H3K27me3 mark is associated with a more differentiated phenotype related to a committed state of leukemic cells.

Histone Protein Expression in AML Patients

To study the role of histones on AML clinical and biological features, we studied the effect of H3K27me3 HIST1 epigenetic silencing on the level of histone linker H1d encoded by HIST1HID. We chose to analyze specifically H1d because its mRNA level is affected by H3K27me3 HIST1 status (data not shown) and it is the leading gene for the mRNA signature (data not shown). In addition, H1 histone subtypes are heterogeneous in amino acid composition²³, which probably reflects a subtype-specific function. First we looked at proportions of total histones and of each histone subtype (data not shown) in chromatin-bound fractions extracted from a series of 12 patient samples (six in each group) using Intensity Based Absolute Quantification (iBAQ) approach. Normalized quantities of total linker histone H1 and core histones H2A, H2B, H3 and H4 were similar in both H3K27me3 HIST1^{high} and H3K27me3 HIST1^{low} patients (data not shown) suggesting that H3K27me3 HIST1^{high} status did not globally modify histone protein abundance.

Yet, when looking at the H1 subtype abundance, we observed that the H1d subtype was decreased in the H3K27me3 HIST1^{high} group (normalized iBAQ value (Log 2)=6.09 vs 4.74; P=0.04) whereas the other H1 subtypes, H1b, Hic H1F0 and H1FX were unaffected (data not shown). These results are consistent with the HIST1H1D mRNA expression decrease observed in AML samples harboring the H3K27me3 HIST1 mark. We confirmed the lower expression of H1d observed in H3K27me3 HIST1^{high} group in comparison to H3K27me3 HIST1^{low} group of patients by western blot using pan H1 and specific H1d antibodies (data not shown).

In conclusion, as a consequence of the presence of an H3K27me3 islet, NPM1mut CN-AML H3K27me3 HIST1^{high} patients express low level of H1d.

H1d Knockdown Confers a More Mature Phenotype in OCI-AML3 Cell Line

To investigate the effect of low H1d expression on AML, we performed H1d knockdown (KD) in OC1-AML3 cells, an AML cell line expressing a NPM1 mutated allele.²⁶ Efficiency and specificity of our KD were assessed by testing the different mRNA H1 subtypes expression by q-PCR (FIG. 2A) and by measuring H1d protein level after doxycycline induction (data not shown). Consequences of H1d KD on differentiation were evaluated by measuring CD11b and CD11c expression in OCI-AML3 after doxycycline induction. H1d KD did not induce a significant increase in CD11b (FIG. 2B) nor CD11c levels (data not shown). As OCI-AML3 can differentiate in vitro in the presence of ATRA, albeit at low efficiency,^{27,28} we further analyzed the effect of H1d KD in combination with ATRA treatment. Two different doses of ATRA (0.5 μM and 1 μM) induced a significant increase in CD11b expression, with a marked increase at 0.5 μM (22.6%±2.5 vs 41%±4.3; P=0.008) (FIG. 2C). We also observed an increase in the proportion of the double positive CD11b/CD11c populations in ATRA-treated H1d KD cells (29.8%±1.3 vs 42.5%±2.1; P=0.003) (FIG. 2B). That result suggests that a lower expression of H1d sensitizes AML cells to ATRA treatment. Next, we assessed the cellular morphology by May-Grunwald Giemsa coloration; at 96 h of ATRA treatment (0.5 μM), cytoplasmic granules that reflects the beginning of a maturation process appeared upon H1d KD (data not shown). Quantification revealed a higher proportion of cells with more than two cytoplasmic granules in the H1d KD (9.7%±2.6 vs 31.5±3.7; P=0.009), (FIG. 2D). Finally, mRNA expression levels of two ATRA-induced genes, CYBB and ITGAM²⁹ were tested under H1d KD condition with ATRA-treatment; H1d down-regulation increased the amplitude of ATRA-induced upregulation of these two genes (FIG. 2E). Altogether these results suggest that down regulation of histone H1D induces ATRA-sensitization, and provide hypotheses to explain the more mature phenotype found in H3K27me3 HIST1^{high} leukemia and suggest that ATRA could be a more efficient differentiating agent in NPM1mut AML with low H1d expression.

Implication of the H3K27meHIST1 Signature in NMP1-Mut CN-AML Progression and Sensitivity to Treatment

We questioned the implication of the H3K27meHIST1 signature in NMP1-mut CN-AML progression and sensitivity to treatment by testing a panel of drugs (78 FDA-approved and/or investigational drug compounds including chemo and epidrugs) on H3K27me3HIST1^{high} versus H3K27me3HIST1^{low} patient cells (FIG. 3A). Our preliminary results obtained with 4 H3K27me3HIST1^{high} and 3 H3K27me3HIST1^{low} AML patient cells suggested that H3K27me3HIST1^{high} condition was more sensitive to chemotherapy (FIG. 3B-D), which reflected the better survival observed in patients (Garciaz, Clin Epigenetics, 2019, 11:141). In addition, we identified 4 interesting drugs that were more effective in H3K27me3HIST1^{high} than in H3K27me3HIST1^{low} AMLs (FIG. 3B-D). Altogether, these results suggest that H3K27me3HIST1^{high} confers vulnerability to AML cells.

We determined the response to chemotherapy according to H3K27me3 status. This was tested in vivo using patient-derived xenograft (PDX) treated with chemotherapeutic drugs (Ara-C). We used secondary transplantation from 1 H3K27me3HIST1low and 2 H3K27me3HIST1high NPM1mut AML patients. Experimental groups of 6 mice/groups of H3K27me3 HIST1high versus H3K27me3 HIST1low were studied. Mice were treated for one week and progression of leukemia was evaluated by counting hCD45+ cells in blood and after euthanasia in bone marrow. Mouse maintenance and experimental procedures were performed in accordance with protocols approved and compliance with policies approved by the local Committee for Animal Experimentation of Marseille (CAE of Provence number 14), France (2-091009).

Response to chemotherapy was obtained in all mice and recovery was in the same range of time for all the PDX tested, independently of H3K27me3HIST1 status. We performed a second round of ARAC treatment and analyzed the presence of leukemic cells 4-day post treatment. Numbers of blasts were variable depending on the patient with an accumulation in the BM of leukemic Blast in one of the PDX. The leukemic initiating cell (LIC) has been shown to be responsible of treatment resistance and relapse. Thus we analyzed the % of LIC in the 2-round ARAC treated mice in comparison to untreated ones (FIG. 4A). Percentage of LIC was identical before and after treatment in the H3K27me3HIST1high PDX while the percentage of LIC was increased in the H3K27me3HIST1low PDX (FIG. 4B). This suggests that ARAC treatment increased the LIC percentage in the leukemia bulk of H3K27me3HIST1low AML samples.

### REFERENCES

Throughout this application, various references describe the state of the art to which this invention pertains. The disclosures of these references are hereby incorporated by reference into the present disclosure.
1. Grimwade D, Ivey A, Huntly B J P. Molecular landscape of acute myeloid leukemia in younger adults and its clinical relevance. Blood. 2016; 127(1):29-41.
2. Falini B, Mecucci C, Tiacci E, et al. Cytoplasmic nucleophosmin in acute myelogenous leukemia with a normal karyotype. N. Engl. J. Med. 2005; 352(3):254-266.
3. Cancer Genome Atlas Research Network. Genomic and epigenomic landscapes of adult de novo acute myeloid leukemia. N. Engl. J. Med. 2013; 368(22):2059-2074.
4. Papaemmanuil E, Gerstung M, Bullinger L, et al. Genomic Classification and Prognosis in Acute Myeloid Leukemia. N. Engl. J. Med. 2016; 374(23):2209-2221.
5. Heath E M, Chan S M, Minden M D, et al. Biological and clinical consequences of NPM1 mutations in AML. Leukemia. 2017; 31(4):798-807.
6. Wouters B J, Delwel R. Epigenetics and approaches to targeted epigenetic therapy in acute myeloid leukemia. Blood. 2016; 127(1):42-52.
7. Sashida G, Iwama A. Multifaceted role of the polycomb-group gene EZH2 in hematological malignancies. Int. J. Hematol. 2017; 105(1):23-30.
8. Morin R D, Johnson N A, Severson T M, et al. Somatic mutations altering EZH2 (Tyr641) in follicular and diffuse large B-cell lymphomas of germinal-center origin. Nat. Genet. 2010; 42(2): 181-185.
9. Ntziachristos P, Tsirigos A, Van Vlierberghe P, et al. Genetic inactivation of the polycomb repressive complex 2 in T cell acute lymphoblastic leukemia. Nat. Med. 2012; 18(2):298-301.
10. Ernst T, Chase A J, Score J, et al. Inactivating mutations of the histone methyltransferase gene EZH2 in myeloid disorders. Nat. Genet. 2010; 42(8):722-726.
11. Ginner S, Oellerich T, Agrawal-Singh S, et al. Loss of the histone methyltransferase EZH2 induces resistance to multiple drugs in acute myeloid leukemia. Nat. Med. 2017; 23(1):69-78.
12. Figueroa M E, Lugthart S, Li Y, et al. DNA methylation signatures identify biologically distinct subtypes in acute myeloid leukemia. Cancer Cell. 2010; 17(1):13-27.
13. Deneberg S, Guardiola P, Lennartsson A, et al. Prognostic DNA methylation patterns in cytogenetically normal acute myeloid leukemia are predefined by stem cell chromatin marks. Blood. 2011; 118(20):5573-5582.
14. Marzluff W F, Gongidi P, Woods K R, Jin J, Maltais L J. The human and mouse replication-dependent histone genes. Genomics. 2002; 80(5):487-498.
15. Tiberi G, Pekowska A, Oudin C, et al. PcG methylation of the HIST1 cluster defines an epigenetic marker of acute myeloid leukemia. Leukemia. 2015; 29(5):1202-1206.
16. Koubi M, Poplineau M, Vernerey J, et al. Regulation of the positive transcriptional effect of PLZF through a non-canonical EZH2 activity. Nucleic Acids Res. 2018;
17. Schlenk R F, Döhner K, Krauter J, et al. Mutations and treatment outcome in cytogenetically normal acute myeloid leukemia. N. Engl. J. Med. 2008; 358(18):1909-1918.
18. Ley T J, Ding L, Walter M J, et al. DNMT3A mutations in acute myeloid leukemia. N. Engl. J. Med. 2010; 363(25):2424-2433.
19. Metzeler K H, Hummel M, Bloomfield C D, et al. An 86-probe-set gene-expression signature predicts survival in cytogenetically normal acute myeloid leukemia. Blood. 2008; 112(10):4193-4201.
20. Kohlmann A, Bullinger L, Thiede C, et al. Gene expression profiling in AML with normal karyotype can predict mutations for molecular markers and allows novel insights into perturbed biological pathways. Leukemia. 2010; 24(6):1216-1220.
21. Bei L, Lu Y, Eklund E A. HOXA9 activates transcription of the gene encoding gp91Phox during myeloid differentiation. J. Biol. Chem. 2005; 280(13):12359-12370.
22. Richard M, Veilleux P, Rouleau M, Paquin R, Beaulieu A D. The expression pattern of the ITIM-bearing lectin CLECSF6 in neutrophils suggests a key role in the control of inflammation. J. Leukoc. Biol. 2002; 71(5):871-880.
23. Bagger F O, Sasivarevic D, Sohi S H, et al. BloodSpot: a database of gene expression profiles and transcriptional programs for healthy and malignant haematopoiesis. Nucleic Acids Res. 2016;44(D1):D917-924.
24. Ng S W K, Mitchell A, Kennedy J A, et al. A 17-gene stemness score for rapid determination of risk in acute leukaemia. Nature. 2016; 540(7633):433-437.
25. Hergeth S P, Schneider R. The H1 linker histones: multifunctional proteins beyond the nucleosomal core particle. EMBO Rep. 2015; 16(11):1439-1453.
26. Quentmeier H, Martelli M P, Dirks W G, et al. Cell line OCI/AML3 bears exon-12 NPM gene mutation-A and cytoplasmic expression of nucleophosmin. Leukemia. 2005; 19(10): 1760-1767.
27. Martelli M P, Gionfriddo I, Mezzasoma F, et al. Arsenic trioxide and all-trans retinoic acid target NPM1 mutant oncoprotein levels and induce apoptosis in NPM1-mutated AML cells. Blood. 2015; 125(22):3455-3465.
28. El Hajj H, Dassouki Z, Berthier C, et al. Retinoic acid and arsenic trioxide trigger degradation of mutated NPM1, resulting in apoptosis of AML cells. Blood. 2015; 125(22):3447-3454.
29. Boutzen H, Saland E, Larrue C, et al. Isocitrate dehydrogenase 1 mutations prime the all-trans retinoic acid myeloid differentiation pathway in acute myeloid leukemia. J. Exp. Med. 2016; 213(4):483-497.
30. Chatterjee A, Rodger E J, Eccles M R. Epigenetic drivers of tumourigenesis and cancer metastasis. Semin. Cancer Biol. 2017;
31. Kernytsky A, Wang F, Hansen E, et al. IDH2 mutation-induced histone and

DNA hypermethylation is progressively reversed by small-molecule inhibition. Blood. 2015; 125(2):296-303.
32. Döhner H, Estey E, Grimwade D, et al. Diagnosis and management of AML in adults: 2017 ELN recommendations from an international expert panel. Blood. 2017; 129(4):424-447.
33. Ivey A, Hills R K, Simpson M A, et al. Assessment of Minimal Residual Disease in Standard-Risk A M L. N. Engl. J. Med. 2016; 374(5):422-433.
34. Li Y, He Y, Liang Z, et al. Alterations of specific chromatin conformation affect ATRA-induced leukemia cell differentiation. Cell Death Dis. 2018;9(2):200.
35. Bertoli S, Picard M, Berard E, et al. Dexamethasone in hyperleukocytic acute myeloid leukemia. Haematologica. 2018;
36. Eppert K, Takenaka K, Lechman E R, et al. Stem cell gene expression programs influence clinical outcome in human leukemia. Nat. Med. 2011; 17(9):1086-1093.
37. Quek L, Otto G W, Garnett C, et al. Genetically distinct leukemic stem cells in human CD34-acute myeloid leukemia are arrested at a hemopoietic precursor-like stage. J.

Exp. Med. 2016; 213(8):1513-1535.
38. Fritz A J, Ghule P N, Boyd J R, et al. Intranuclear and higher-order chromatin organization of the major histone gene cluster in breast cancer. J. Cell. Physiol. 2017;
39. Medrzycki M, Zhang Y, McDonald J F, Fan Y. Profiling of linker histone variants in ovarian cancer. Front. Biosci. Landmark Ed. 2012; 17:396-406.
40. Bauden M, Kristl T, Sasor A, et al. Histone profiling reveals the H1.3 histone variant as a prognostic biomarker for pancreatic ductal adenocarcinoma. BMC Cancer. 2017;17(1):810.
41. Izzo A, Schneider R. The role of linker histone H1 modifications in the regulation of gene expression and chromatin dynamics. Biochim. Biophys. Acta. 2016; 1859(3):486-495.
42. Sancho M, Diani E, Beato M, Jordan A. Depletion of human histone H1 variants uncovers specific roles in gene expression and cell growth. PLoS Genet. 2008;4(10):e1000227.
43. Medrzycki M, Zhang Y, Zhang W, et al. Histone h1.3 suppresses h19 noncoding RNA expression and cell growth of ovarian cancer cells. Cancer Res. 2014; 74(22):6463-6473.
44. Tones C M, Biran A, Burney M J, et al. The linker histone H1.0 generates epigenetic and functional intratumor heterogeneity. Science. 2016;353(6307):.
45. Warrell R P, Frankel S R, Miller W H, et al. Differentiation therapy of acute promyelocytic leukemia with tretinoin (all-trans-retinoic acid). N. Engl. J. Med. 1991;324(20): 1385-1393.
46. Balusu R, Fiskus W, Rao R, et al. Targeting levels or oligomerization of nucleophosmin 1 induces differentiation and loss of survival of human AML cells with mutant NPM1. Blood. 2011; 118(11):3096-3106.
47. Tassara M, Darner K, Brossart P, et al. Valproic acid in combination with all-trans retinoic acid and intensive therapy for acute myeloid leukemia in older patients. Blood. 2014; 123 (26):4027-4036.
48. Schenk T, Chen W C, Ginner S, et al. Inhibition of the LSD1 (KDM1A) demethylase reactivates the all-trans-retinoic acid differentiation pathway in acute myeloid leukemia. Nat. Med. 2012; 18(4):605-611.
49. Baik H, Boulanger M, Hosseini M, et al. Targeting the SUMO pathway primes all-trans retinoic acid-induced differentiation of non-promyelocytic acute myeloid leukemias. Cancer Res. 2018;
Pratiksha I. Thakore, Jennifer B. Kwon, Christopher E. Nelson, Douglas C. Rouse, Matthew P. Gemberling, Matthew L. Oliver & Charles A. Gersbach. RNA-guided transcriptional silencing in vivo with S. aureus CRISPR-Cas9 repressors.

1. (canceled)

2. A method of sensitizing cancerous cells in a patient to therapeutic compounds used to treat AML, comprising
administering to the patient an amount of an H1d inhibitor sufficient to sensitize the cancerous cells to the therapeutic compounds.

3-4. (canceled)

5. A method for treating AML in a patient in need thereof, comprising administering to the patient a therapeutically effective amount of an inhibitor of H1d.

6-8. (canceled)

9. A method of treatment of an AML in a patient in need thereof comprising the step of:
a. determining if the patient as has a good or a bad prognosis by
i) determining, in a sample obtained from the patient, the expression level of at least one gene selected in the group consisting in CYBB, FCN1, CLEC4 and ITGAM; and
ii) comparing the expression level of the at least one gene determined at step i) with a predetermined reference value; and
when the expression level determined at step i) is higher than the predetermined reference value,
b. administering to said patient a therapeutically effective amount of a compound useful for the treatment of AML.

10-12. (canceled)

13. A method of treating an AML in a patient in need thereof comprising:
a. determining if the patient is a good responder to a chemotherapeutic agent by
i) determining in a sample obtained from the patient the histone tri-methylation profile level of H3K27,
ii) comparing the histone tri-methylation profile level of H3K27 determined at step i) with a predetermined reference value and,
when the histone tri-methylation profile level determined at step i) is higher than the predetermined reference value,
b. administrating to said patient a therapeutically effective amount of the chemotherapeutic agent.

14. The method of claim 5, wherein the AML is a Cytogenetically normal AML (CT-AML), an acute promyelocytic leukemia (APL), an acute myeloid leukemia with trisomy 8 or an acute leukemia with MLL translocations.

15. The method of claim 5, wherein the H1d inhibitor is all-trans retinoic acid (ATRA), gemtuzumab or ozogamicin; or a combination of methotrexate, mercaptopurine and ATRA; or a demethylating agent.

16. The method of claim 5, further comprising administering to the patient a therapeutic compound used to treat AML or allograft.

17. The method of claim 16 wherein the H1d inhibitor and the compound used to treat AML or allograft are administered simultaneously, separately or sequentially.

18. The method claim 13, wherein the patient is suffering from CN-AML and has a NPM1 mutation.

19. The method claim 13, wherein the chemotherapeutic agent is selected from the group consisting of cytarabine (araC), volasertib, tozasertib (VX-680), nutlin 3 and olaparib.
